(19) United States
(12) Patent Application Publication
(10) Pub. No.: US 2008/0045482 A1
(43) Pub. Date: Feb. 21, 2008

(54) COMPOSITIONS AND METHODS FOR THE TREATMENT OF VIRAL INFECTIONS

(75) Inventors: Joan Robbins, San Diego, CA (US); Shani Waninger, Carlsbad, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Adventrx Pharmaceuticals, Inc., San Diego, CA (US)

(21) Appl. No.: 11/627,062

(22) Filed: Jan. 25, 2007

Related U.S. Application Data
(60) Provisional application No. 60/762,832, filed on Jan. 27, 2006.

(51) Int. Cl. A61K 31/66; A61P 31/12

(52) U.S. Cl. 514/120

(57) ABSTRACT

The present invention provides compositions and methods for inhibiting the replication of influenza virus by administering thiophosphonoformic acid alone or in combination with a neuraminidase inhibitor, for example, oseltamivir.

### CROSS-REFERENCES TO RELATED APPLICATIONS

This application claims the benefit of U.S. Provisional Application No. 60/762,832, filed on Jan. 27, 2006, the entire contents of which is hereby incorporated herein by reference in its entirety.

### FIELD OF THE INVENTION

The present invention provides novel compounds and methods for the treatment of viral infections, in particular, infections caused by the influenza virus.

### BACKGROUND OF THE INVENTION

Recently, the attention of health authorities around the world has focused on the concern that a pandemic may break out as a result of a mutation of a form of Influenza A currently transmitted only by birds (the so called avian or “bird flu”). Like all viruses, this strain of Influenza A (H5N1) is highly susceptible to mutation, and the concern is that a mutation will occur that will allow the virus to be transmitted between humans. In such event, it will be exceedingly difficult to prevent widespread infection across large populations, and a world-wide pandemic may occur.

In the face of such a threat, significant research is being conducted around the world to design a vaccine that would effectively prevent entry of the virus into human cells. Unfortunately, designing such a vaccine is fraught with enormous difficulties, since vaccines by their nature are engineered to bind to viral envelope proteins, and these are particularly susceptible to mutation. Thus, it is impossible to know at this time the exact form of the mutation that will occur that will allow this form of Influenza A to enter human cells.

In the absence of an effective vaccine, or even should a vaccine be provided, it will still be necessary to provide methods of treatment for individuals and populations that will become infected with strains of Influenza A, including “bird flu.” The present invention provides such methods of treatment as well as compositions that are useful for the treatment of such infections.

### BRIEF SUMMARY OF THE INVENTION

In a first aspect, the invention provides methods of inhibiting influenza viral replication in a subject infected with influenza virus by administering to the subject a therapeutically effective amount of a composition comprising thiophosphonoformic acid (TPFA). The influenza virus can be Influenza A or Influenza B strains.

In a further aspect, the invention provides methods of inhibiting influenza viral replication in a subject infected with influenza virus by co-administering to the subject a therapeutically effective amount of thiophosphonoformic acid (TPFA) and a therapeutically effective amount of oseltamivir. In some embodiments, the subject is administered amounts of thiophosphonoformic acid and oseltamivir to synergistically lower the viral load in the subject infected with influenza. In some embodiments, the thiophosphonoformic acid and oseltamivir are administered together in subtherapeutic doses of each agent.

With respect to the embodiments of the methods, in some embodiments, the influenza virus is Influenza A selected from the group comprising H1N1, H3N2, H3N8, H5N1 and H5N2. In some embodiments, the influenza virus is Influenza B.

In some embodiments, the composition comprises the trisodium salt, dihydrate form of TPFA.

In some embodiments, the composition is administered orally. In some embodiments, the composition is administered by parenteral injection.

In some embodiments, the TPFA and oseltamivir are administered at the same time.

In another aspect, the invention provides a composition comprising a therapeutically effective amount of thiophosphonoformic acid and a therapeutically effective amount of oseltamivir. In some embodiments, the compositions comprise amounts of thiophosphonoformic acid and oseltamivir to synergistically lower the viral load in the subject infected with influenza.

### DEFINITIONS

The term “therapeutically effective amount” refers to an amount of the one or more active agents sufficient to achieve the desired effect, here inhibition of replication of influenza virus, without causing undesirable side effects.

The term “subject” refers to any animal susceptible to influenza virus, including avians and mammals. Mammalian subjects include humans, non-human primates, agricultural mammals (e.g., bovines, equines, ovines, porcines, etc.), domestic or companion mammals (e.g. canines, felines, etc.) and laboratory animals (e.g., rabbits, rats, mice, hamsters, etc.). Treatable avian subjects include chickens, ducks, geese, turkeys, etc.

The term “synergistic” or “synergy” interchangeably refer to the interaction of two or more agents so that their combined effect is greater than the sum of their individual effects. Synergistic drug interactions can be determined using the median effect principle (see, Chou and Talalay (1984) Adv Enzyme Regul 22:27 and Synergism and Antagonism in Chemotherapy, Chou and Rideout, eds., 1996, Academic, pp. 61-102) and quantitatively determined by combination indices using the computer program Calcusyn (Chou and Hayball, 1996, Biosoft, Cambridge, Mass.). See also, Reynolds and Maurer, Chapter 14 in Methods in Molecular in Medicine, vol. 110: Chemosensitivity, Vol. 1: In Vitro Assays, Blumenthal, ed., 2005, Humana Press. Combination indices (CI) quantify synergy, summation and antagonism as follows: CI<1 (synergy); CI=1 (summation); CI>1 (antagonism). A CI value of 0.7-0.9 indicates moderate to slight synergism. A CI value of 0.3-0.7 indicates synergism. A CI value of 0.1-0.3 indicates strong synergism. A CI value of <0.1 indicates very strong synergism.

The phrase “sufficient to synergistically lower the viral load or viral titer” refers an amount and proportion of two or more active agents such that their combined effects lower viral titer or viral load greater than the sum of the individual active agents. The synergistic effect on viral load is measured in vitro or in vivo using test known in the art, including the tests described herein (e.g. viral plaque reduction assay or influenza (A or B strains) nucleocapsid protein antigen capture ELISA). Sufficient amounts and proportions of the two or more active agents to operate synergistically to lower viral load can be determined by the amounts administered or by the concentrations of the active agents in a body fluid (e.g., blood, serum, plasma, saliva, urine).

The term “thiophosphonoformic acid” or “TPFA” interchangeably refer to a thio-analogue of phosphonoformic acid having the following chemical formula (HO)₂P(S)COOH:

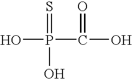

The term “co-administer” refers to the simultaneous presence of two active agents in the blood of an individual. Active agents that are co-administered can be concurrently or sequentially delivered.

### DETAILED DESCRIPTION

1. Introduction

Thiophosphonoformic acid (TPFA) is an analogue of phosphonoformic acid (foscamet). It has been surprisingly discovered that TPFA has particularly inhibitory activity against influenza virus, including Influenza A and Influenza B strains. While foscarnet has been shown to have anti-influenza activity, the literature indicates that foscamet analogues are not predictable with regard to their antiviral properties. Furthermore, the analogues do not act as equivalents of foscamet. For example, in U.S. Pat. No. 5,183,812, foscarnet and TPFA were not equivalently effective in viral polymerase inhibition of Herpes Simplex Virus Type I and II, Epstein-Barr Virus and Herpes Virus 6. Moreover, Strid, et al., Chemotherapy (1989) 35:69-76 synthesized numerous foscarnet analogues, most of which did not demonstrate significant Influenza A inhibitory activity in viral plaque reduction assays (i.e., IC₉₀ concentrations of 500 μM or more). Strid did not test TPFA.

Furthermore, the inventors have found that TPFA unpredictably exhibits inhibitory activity against some viruses, but not others. For example, we have found that TPFA has inhibitory activity against Human Immunodeficiency Virus-1 (HIV-1), HIV-2, Herpes Simplex Virus-1 (HSV-1), HSV-2, Human Influenza Virus (H1N1, H3N2), Equine Influenza Virus (H3N8) and Avian Influenza Virus (H5N2). However, TPFA does not have inhibitory activity against Adenovirus type-1 (AdV-1, strain 65089), Punta Torro Virus (strain Adames), Pichinde Virus (strain 4763), Vaccinia Virus (strain WR), Venezuelan Equine Encephalitis Virus (VEE, Trinidad strain), West Nile Virus (WNV, strain New York), or Yellow Fever Virus (17D strain).

Our data demonstrates that TPFA consistently exhibits inhibitory activity against Influenza type A (“Influenza A”) and the Influenza type B (“Influenza B”) virus strains. Accordingly, the present invention provides pharmaceutical compositions and methods for the treatment of viral infections, particularly those caused by the Influenza type A virus (“Influenza A”) or the Influenza type B virus (“Influenza B”). The compositions and methods of the invention that are effective in treating influenza infections inhibit the replication of the influenza virus once the virus has entered infected cells.

The compositions of the invention comprise a pharmaceutically acceptable form of thiophosphonoformic acid (TPFA) and a pharmaceutically acceptable carrier. The methods of the invention comprise administering to an individual infected with influenza virus a pharmaceutically effective amount of TPFA, either alone or in combination with one or more additional antiviral drugs, for example one or more neuraminidase inhibitors, for example, oseltamivir.

2. Compositions

The present invention provides pharmaceutical compositions comprising a therapeutically effective amount of thiophosphonoformic acid and a pharmaceutically acceptable carrier. The present invention also provides pharmaceutical compositions comprising a mixture of a therapeutically effective amount of thiophosphonoformic acid and an effective amount of one or more neuraminidase inhibitors, particularly oseltamivir, such that the amounts and proportions of the active agents synergistically lower viral load in an individual.

a. Active Agents

i. Thiophosphonoformic Acid

As used herein, thiophosphonoformic acid (TPFA) refers to an active agent having the following chemical formula:

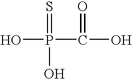

Thiophosphonates and methods for their synthesis are disclosed, for example, in U.S. Pat. Nos. 5,072,032; 5,183,812; 6,147,244 and 6,284,909, the entire contents of which are hereby incorporated herein by reference for all purposes. Thiophosphonoformic acid can also be synthesized through commercial service providers, for example, Natland International Corp., Morrisville, N.C.; Custom Synthesis Inc., Delray Beach, Fla.; D Pharm Innovative Biopharmaceuticals, Princeton, N.J. and Chemshop, Weert, The Netherlands.

One pharmaceutically acceptable form of TPFA (manufactured under license from the University of Southern California, trade name Thiovir™) has the following structure:

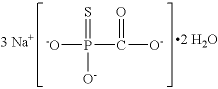

This trisodium salt, dihydrate, form of TPFA is a white to off white powder with the molecular formula Na₃CO₄PS.2H₂O and molecular weight of 244.042. The aqueous solubility is 40.8 g/100 mL at 37° C. in distilled water. The pH in water is approximately 9.

In the body, TPFA is metabolized to phosphonoformic acid and thiophosphonic acid. Phosphonoformic acid, trisodium salt (PFA) is the active ingredient in the antiviral drug foscarnet (manufactured by AstraZenica under the proprietary name FOSCAVIR). Unmetabolized TPFA also exhibits anti-viral activity.

ii. Neuraminidase Inhibitors

Neuraminidase inhibitors are a class of antiviral drugs whose mode of action relies on blocking the function of viral neuraminidase protein, thus preventing the virus from budding from the host cell. Unlike the M2 inhibitors, which work only against the Influenza A, neuraminidase inhibitors act against both Influenza A and B.

Exemplified neuraminidase inhibitors that find use in the present compositions include, for example, Oseltamivir, Zanamivir and Peramivir. Oseltamivir, (3R,4R,5S)-4-acetylamino-5-amino-3-(1-ethylpropoxy)-1-cyclohexene-1-carboxylic acid ethyl ester (CAS number 196618-13-0), was developed by Gilead Sciences and is currently marketed by Hoffmann-La Roche (Roche) under the trade name Tamiflu. Zanamivir, 5-acetamido-4-guanidino-6-(1,2,3-trihydroxypropyl)-5,6-dihydro-4H-pyran-2-carboxylic acid, (CAS number 139110-80-8), is currently marketed by GlaxoSmithKline under the trade name Relenza. Peramivir, 3-(1′-acetylamino-2′-ethyl)butyl-4-((aminoimino)methyl)amino-2-hydroxycyclopentane-1-carboxylic acid (CAS number 330600-85-6), is owned by BioCryst Pharmaceuticals, Birmingham, Ala.

In preferred embodiments, the compositions of the invention comprise a therapeutically effective amount of thiophosphonoformic acid and a therapeutically effective amount of oseltamivir.

A thiophosphonoformic acid and neuraminidase inhibitor combination of this invention can be incorporated into a variety of formulations for therapeutic administration. More particularly, a combination of the present invention can be formulated into pharmaceutical compositions, together or separately, by formulation with appropriate pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of a thiophosphonoformic acid and neuraminidase inhibitor combination can be achieved in various ways, including oral, buccal, parenteral, intravenous, intradermal (e.g., subcutaneous, intramuscular), topical, transdermal, etc., administration. Moreover, the compound can be administered in a local rather than systemic manner, for example, in a depot or sustained release formulation. In a preferred embodiment, the invention provides for a pharmaceutical composition comprised of thiophosphonoformic acid and one or more neuraminidase inhibitors for oral delivery (e.g., a tablet, a capsule, a powder, a liquid, a syrup, etc.).

Suitable formulations for use in the present invention can be found in Remington: The Science and Practice of Pharmacy, 21st Edition, 2005, University of the Sciences in Philadelphia (USIP), Lippincott, Williams & Wilkins. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

In one preferred embodiment, a thiophosphonoformic acid and neuraminidase inhibitor combination is prepared for delivery in a sustained-release, controlled release, extended-release, timed-release or delayed-release formulation, for example, in semipermeable matrices of solid hydrophobic polymers containing the therapeutic agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients (see, for example, Huang, et al. Drug Dev. Ind. Pharm. 29:79 (2003); Pearnchob, et al. Drug Dev. Ind. Pharm. 29:925 (2003); Maggi, et al. Eur. J. Pharm. Biopharm. 55:99 (2003); Khanvilkar, et al., Drug Dev. Ind. Pharm. 228:601 (2002); and Schmidt, et al., Int. J. Pharm. 216:9 (2001)). Sustained-release delivery systems can, depending on their design, release the compounds over the course of hours or days, for instance, over 4, 6, 8, 10, 12, 16, 20, 24 hours or more. For example, sustained release formulations can be prepared using naturally-occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

The sustained or extended-release formulations can also be prepared using natural ingredients, such as minerals, including titanium dioxide, silicon dioxide, zinc oxide, and clay (see, U.S. Pat. No. 6,638,521, herein incorporated by reference). Exemplified extended release formulations that can be used in delivering a thiophosphonoformic acid and neuraminidase inhibitor combination of the present invention include those described in U.S. Pat. Nos. 6,635,680; 6,624,200; 6,613,361; 6,613,358, 6,596,308; 6,589,563; 6,562,375; 6,548,084; 6,541,020; 6,537,579; 6,528,080 and 6,524,621, each of which is hereby incorporated herein by reference. Controlled release formulations of particular interest include those described in U.S. Pat. Nos. 6,607,751; 6,599,529; 6,569,463; 6,565,883; 6,482,440; 6,403,597; 6,319,919; 6,150,354; 6,080,736; 5,672,356; 5,472,704; 5,445,829; 5,312,817 and 5,296,483, each of which is hereby incorporated herein by reference. Those skilled in the art will readily recognize other applicable sustained release formulations.

For oral administration, a thiophosphonoformic acid and neuraminidase inhibitor combination can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents can be added, such as a cross-linked polyvinyl pyrrolidone, agar, or alginic acid or a salt thereof such as sodium alginate.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

The compounds can be formulated for parenteral administration by injection, e.g., by bolus injection or continuous infusion. For injection, a thiophosphonoformic acid and neuraminidase inhibitor combination can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, a combination of the invention can be formulated in aqueous solutions, preferably in physiologically compatible buffers such as Hanks's solution, Ringer's solution, or physiological saline buffer. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

For topical administration, the agents are formulated into ointments, creams, salves, powders and gels.

Systemic administration can also be by transmucosal or transdermal means. For transmucosal or transdermal administration, penetrants appropriate to the barrier to be permeated are used in the formulation. In one embodiment, the transdermal delivery agent can be, for example, DMSO, urea, 1-methyl-2-pyrrolidone, oleic acid, or a terpene (e.g., 1-menthol, d-limonene, RS-(+/−)-beta-citronellol, geraniol). Further percutaneous penetration enhancers are described, for example, in Percutaneous Penetration Enhancers, Smith and Maibach, eds., 2^{nd} edition, 2005, CRC Press. Transdermal delivery systems can include, e.g., patches. For transmucosal administration, penetrants appropriate to the barrier to be permeated are used in the formulation. Such penetrants are generally known in the art. Exemplified transdermal delivery formulations that can find use in the present invention include those described in U.S. Pat. Nos. 6,589,549; 6,544,548; 6,517,864; 6,512,010; 6,465,006; 6,379,696; 6,312,717 and 6,310,177, each of which are hereby incorporated herein by reference.

For buccal administration, the compositions can take the form of tablets or lozenges formulated in conventional manner.

In addition to the formulations described previously, a thiophosphonoformic acid and neuraminidase inhibitor combination of the present invention can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions suitable for use in the present invention include compositions wherein the active ingredients are contained in a therapeutically effective amount. The amount of composition administered will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein. Generally, an efficacious or effective amount of a thiophosphonoformic acid and neuraminidase inhibitor combination is determined by first administering a low dose of one or both active agents and then incrementally increasing the administered dose or dosages until a desired effect of reduced viral titer is observed in the treated subject, with minimal or no toxic side effects. Applicable methods for determining an appropriate dose and dosing schedule for administration of a combination of the present invention are described, for example, in Goodman and Gilman's The Pharmacological Basis of Therapeutics, 11th Edition, 2006, Brunton, et al., eds., McGraw-Hill and in Remington: The Science and Practice of Pharmacy, 21st Edition, supra.

Dosage amount and interval can be adjusted individually to provide plasma levels of the active compounds which are sufficient to maintain therapeutic effect. Preferably, therapeutically effective serum levels will be achieved by administering single daily doses, but efficacious multiple daily dose schedules are included in the invention. In cases of local administration or selective uptake, the effective local concentration of the drug may not be related to plasma concentration. One having skill in the art will be able to optimize therapeutically effective local dosages without undue experimentation.

3. Methods

The present methods are directed to the inhibiting the replication of an influenza virus, including Influenza A and Influenza B virus strains, by administration of a therapeutically effective amount of thiophosphonoformic acid (TPFA). In some embodiments, the TPFA is co-administered with a therapeutically effective amount of a neuraminidase inhibitor, particularly oseltamivir. When TPFA is co-administered with one or more neuraminidase inhibitors, the TPFA and neuraminidase inhibitor can be delivered concurrently or sequentially, so long as both the thiophosphonoformic acid and the neuraminidase inhibitor are concurrently present in the blood.

a. Routes of Administration

As discussed above, thiophosphonoformic acid and one or more neuraminidase inhibitors can be independently co-administered by any appropriate route of administration. The active ingredients can be administered by the same or different routes of administration, as appropriate. In some embodiments, at least one of the active ingredients is administered orally. In some embodiments, the combination of active ingredients is concurrently orally administered. In some embodiments, at least one of the active ingredients is administered parenterally, for example, intravenously, intramuscularly, subcutaneously, topically, intravaginally, rectally, intranasally, inhalationally, intrathecally, intraocularly.

A combination of thiophosphonoformic acid and one or more neuraminidase inhibitors can be administered to a subject, e.g., a human patient, a domestic animal (e.g., a cat or a dog), an agricultural animal (e.g., a horse, a cow, a sheep, a goat, a pig, a chicken, a duck, a goose, a turkey), independently or together in the form of their pharmaceutically acceptable salts, or in the form of a pharmaceutical composition where the compounds are mixed with suitable carriers or excipients in a therapeutically effective amount, e.g., at doses effective to synergistically effect desired reduction in viral load or viral titer.

b. Dosing

As discussed above, those of skill in the art will recognize that that appropriate doses of thiophosphonoformic acid alone and with one or more neuraminidase inhibitors will depend on several factors, including without limitation, the selected route of administration, the age, weight and prognosis of the patient, the progression of the illness, etc. An efficacious or effective amount of a thiophosphonoformic acid alone and with one or more neuraminidase inhibitor combination can be determined by first administering a low dose of one or both active agents and then incrementally increasing the administered dose or dosages until a desired effect of reduced viral titer is observed in the treated subject, with minimal or no toxic side effects. General considerations for dosing and formulation of thiophosphonoformic acid and one or more neuraminidase inhibitors, individually or combined, can be found in Goodman and Gilman's the Pharmacological Basis of Therapeutics, Goodman, et al., eds., 11^{th} Edition, 2006, McGraw-Hill and Remington: The Science and Practice of Pharmacy, 21st Edition, 2005, University of the Sciences in Philadelphia, Eds., Lippincott Williams & Wilkins.

Thiophosphonoformic acid can be administered in an amount of from about 2 mg/kg to about 100 mg/kg per day, although the doses can be more or less, depending on the route of administration. For example, the doses can be less if the TPFA is administered intravenously. In some embodiments, the thiophosphonoformic acid is administered in an amount of from about 20 mg/kg to about 75 mg/kg per day. In some embodiments, the thiophosphonoformic acid is administered in an amount of about 5, 10, 15, 20, 25, 30, 35, 40, 45, 50, 55, 60, 65, 70, 75 mg/kg per day, or any integer within the range of 2-100 mg/kg per day.

The one or more neuraminidase inhibitors can be administered in doses according to those approved by FDA, although the doses can be less. Approved doses for neuraminidase inhibitors can be found, for example, in the FDA Orange Book, available on the worldwide web at fda.gov/cder/ob/default.htm. For example, approved oral doses for approved neuraminidase inhibitors are as set forth in the table below. Tablets or capsules of neuraminidase inhibitors can be administered one, two, three, four or five times per day, as appropriate.

Doses of Approved Neuramindase Inhibitors

| Neuraminidase | |
|---|---|
| Inhibitor | Dose (mg) |
| oseltamivir | 75 mg per table |
| zanamivir | 5 mg (inhalational powder) |

In some embodiments, at least one of thiophosphonoformic acid and the one or more neuraminidase inhibitors are administered in subtherapeutic doses of the individual active agents. A subtherapeutic dose refers to an amount of an individual active agent that is insufficient to produce an antiviral effect, as measured in vivo or in vitro, using one of the assays described herein. A subtherapeutic dose also refers to a dose amount that is 80% or less of the smallest reference dose amount of an approved active agent. Reference dose amounts are available to those of skill in the art, for example, in Goodman and Gilman's, supra and in the Physician's Desk Reference, 2006, Thompson Publishing. Without being bound to theory, the synergistic antiviral effects of a combination of thiophosphonoformic acid and one or more neuraminidase inhibitors can allow for administration of a subtherapeutic dose of at least one of thiophosphonoformic acid and the one or more neuraminidase inhibitors. Administration of subtherapeutic doses of thiophosphonoformic acid or the neuraminidase inhibitors individually does not produce a significant antiviral effect. However, administration of a subtherapeutic dose of at least one active agent in a combination of thiophosphonoformic acid and one or more neuraminidase inhibitors can produce an antiviral effect, with a lower risk of side effects.

Dosages of thiophosphonoformic acid and/or the one or more neuraminidase inhibitors also can be expressed in term of dose reduction index (DRI). Dose reduction index is a determination of the fold dose reduction allowed for each drug when given in synergistic combination, as compared with the concentration of a single agent that is needed to achieve the same effect level. The synergistic antiviral effects between thiophosphonoformic acid and one or more neuraminidase inhibitors provides for a DRI for thiophosphonoformic acid that is at least about 10, 50, 100, 150, 200, 250, 300, or more. The synergistic antiviral effects between thiophosphonoformic acid and one or more neuraminidase inhibitors provides for a DRI for the one or more neuraminidase inhibitors that is at least about 2, 5, 10, 20, 50, 100, or more.

The combination of thiophosphonoformic acid and one or more neuraminidase inhibitors are administered in amounts such that the molar ratios of the active ingredients allow for synergistic antiviral effects. The thiophosphonoformic acid can be administered at an equimolar ratio to the one or more neuraminidase inhibitors (1:1 molar ratio). The thiophosphonoformic acid can be administered at a molar ratio of about 1:2, 1:5, 1:8, 1:10, or less, to the one or more neuraminidase inhibitors, for example, at any molar ratio from about 1:1 to about 1:100 (TPFA:neuraminidase inhibitor), for example, about 1:15, 1:20, 1:50, 1:100.

The combination of thiophosphonoformic acid and one or more neuraminidase inhibitors are administered in amounts such that the combination index value (CI), determined for example, in a viral plaque reduction assay or a capsid nucleoprotein antigen capture ELISA, is less than 1.0. Preferably, the combination of thiophosphonoformic acid and one or more neuraminidase inhibitors are administered in amounts such that the combination index value is less than about 0.9. More preferably, the combination of thiophosphonoformic acid and one or more neuraminidase inhibitors are administered in amounts such that the combination index value is less than about 0.6.

c. Scheduling

The thiophosphonoformic acid alone or with the one or more neuraminidase inhibitors can be administered concurrently or independently, one, two, three, four or more times in a 24-hour period, as needed. In one embodiment, the thiophosphonoformic acid and one or more neuraminidase inhibitors are administered concurrently. In one embodiment, the thiophosphonoformic acid and one or more neuraminidase inhibitors are administered concurrently once daily, for example, in a sustained-release or delayed release formulation. In one embodiment, the thiophosphonoformic acid and one or more neuraminidase inhibitors are administered concurrently multiple times daily, for example, two, three, or four times daily.

The TPFA alone and with one or more neuraminidase inhibitors are generally administered therapeutically, after symptoms of influenza are detected. However, in certain instances, the one or more active agents can be administered prophylactically, for example, after an individual has been exposed to an influenza virus. The TPFA alone and with one or more neuraminidase inhibitors are generally administered until the symptoms of influenza subside. For example, the TPFA alone and with one or more neuraminidase inhibitors can be administered for 2 days, 3 days, 5 days, 7 days, or longer if necessary, for example, 10 days, 14 days, 21 days or longer.

4. Assays for Synergistic Activity

The following assays can be used to determine inhibition of viral replication effected by thiophosphonoformic acid alone and synergistic antiviral activity of combinations of thiophosphonoformic acid and one or more neuraminidase inhibitors.

a. In vitro

Amounts and proportions of combinations of thiophosphonoformic acid and one or more neuraminidase inhibitors that synergistically reduce viral titer can be determined using in vitro assays.

i. Viral Plaque Reduction Assay

Mammalian cells (e.g., MDCK cells) are seeded at an appropriate density (e.g., about 7×10⁴ per cm²) in a multiwell plate (e.g. a 6-well, 12-well, 24-well, 48-well, or 96-well plate, as desired). Cells are accommodated in culture conditions (e.g., 37° C., 5% CO₂) for about 8-12 hours (e.g., overnight). The culture media is replaced with serum free media and the cells are exposed to the one or more active agents (e.g., TPFA alone or with one or more neuraminidase inhibitors) for about 1 hour prior to adsorption of virus. Virus is added at a multiplicity of infection (MOI) in the range of about 0.02 to about 0.05 for about 2-3 hours in the presence of about 0.5 g/ml trypsin (e.g., 34° C., 5% CO₂).

Following virus adsorption, cells are overlaid with serum-free media, trypsin, and agarose (e.g., about 0.4%) containing the one or more active agents, and incubated for about another 48-72 hours. Plaques are counted from triplicate wells. The 50% (or 75% or 90%) inhibitory concentration, IC₅₀ (or IC₇₅ or IC₉₀, respectively), is determined from a plot of percent inhibition versus log₁₀ drug concentration using dose-effect analysis software, using, for example, Calcusyn (Biosoft, Cambridge, UK).

ii. Influenza Nucleocapsid Protein Antigen Capture ELISA

Mammalian cells (e.g., MDCK cells) are seeded at an appropriate density (e.g., about 7×10⁴ per cm²) in a multiwell plate (e.g. a 6-well, 12-well, 24-well, 48-well, or 96-well plate, as desired). Cells are accommodated in culture conditions (e.g., 37° C., 10% CO₂) for about 8-12 hours (e.g., overnight). The culture media is replaced with serum free media and the cells are exposed to the one or more active agents (e.g., TPFA alone or with one or more neuraminidase inhibitors) for about 1 hour prior to adsorption of virus. Virus is added at a multiplicity of infection (MOI) in the range of about 0.02 to about 0.05 for about 2-3 hours in the presence of about 1.0 g/ml trypsin (e.g., 33.5° C., 5% CO₂).

The cells are washed, resuspended in fresh serum free media containing the one or more test agents, and incubated about another 24 hours. The supernatants are harvested and influenza nucleocapsid protein detected by quantitative antigen capture ELISA (Virusys, Sykesville, Md.). The 50% (or 75% or 90%) inhibitory concentration, IC₅₀ (or IC₇₅ or IC₉₀, respectively), is determined from a plot of percent inhibition versus log₁₀ drug concentration using dose-effect analysis software, using, for example, Calcusyn (Biosoft, Cambridge, UK).

### EXAMPLES

The following examples are offered to illustrate, but not to limit the claimed invention.

Materials and Methods

The following materials and methods were used in the examples provided herein.

Cell Lines and Virus

MDCK cells were obtained from ATCC (Manassas, Va.) and maintained in DMEM medium containing 1 mM sodium pyruvate, 2 mM glutamine, 500 IU/ml pen-strep, 10% fetal bovine serum. Influenza strains A/California/2935/03 (H1N1), A/NorthDakota/2463/03 (H3N2) and Influenza B/1060/03 were obtained from Virapur (San Diego, Calif.) and A/Duck/Pennsylvannia/10218/84 (H5N2) and A/Equine 1/Prague/1/56 (H3N8) were from ATCC.

Drugs

TPFA in the form of Thiovir™ was produced at Chemshop (Weert, Netherlands). Foscarnet was obtained from Sigma-Aldrich (St Louis, Mo.).

Influenza A Nucleocapsid Protein Antigen Capture ELISA

MDCK cells were seeded in 48-well plates at a density of 7×10⁴ cells per cm² and incubated overnight at 37° C., 10% CO₂. Media was replaced with serum free DMEM and cells were pretreated with test article for 60 minutes prior to adsorption of virus at multiplicity of infection (MOI) 0.02 for 2 hrs in the presence of 1 μg/ml TPCK-trypsin at 33.5° C., 10% CO₂. Cells were washed 3 times with PBS and resuspended in fresh serum free media plus test article. Incubation at 33.5° C., 10% CO₂ was continued for 24 hours. Supernatants were removed and Influenza A nucleocapsid protein detected by quantitative antigen capture ELISA (Virusys, Sykesville, Md.).

Viral Plaque Reduction

Viral plaque reduction assays were performed at Virapur (San Diego, Calif.). MDCK cells were seeded at approximately 7×10⁴ cells per cm² in a 6-well plate and incubated overnight at 37° C. in 5% CO₂. Media was replaced with serum free DMEM and cells were pretreated with drug at indicated concentrations for 60 minutes prior to adsorption of virus at MOI 0.02-0.05 for 2-3 hours in the presence of 0.5 μg/ml TPCK-trypsin at 34° C., 5% CO₂. Cells were overlaid with serum free DMEM, 0.5 μg/ml TPCK trypsin, 0.4% agarose, incubation continued for 48-72 hours and plaques scored.

Data Analysis

The 50% inhibitory concentration, IC₅₀, was determined from a plot of percent inhibition vs. log10 drug concentration using dose-effect analysis software, Calcusyn (Biosoft, Cambridge, UK). Combination drug data was analyzed by multiple drug dose-effect calculations using median effect methods (Chou, T. C. and Talalay, P. (1984) Adv. Enzyme Regul. 22, 27-55) and Calcusyn software.

Results

### Example 1

### Inhibition of Influenza A Virus Replication by Thiophosphonoformic Acid

The following example demonstrates the effective inhibition of Influenza A virus replication by TPFA against all strains tested.

Activity of Thiovir Against Influenza A Virus from Multiple Species

Thiovir antiviral activity was evaluated against two strains of human influenza, A/California/2935/03 (H1N1) and A/NorthDakota/2463/03 (H3N2), equine Influenza A/Equine1/Prague/1/56 (H3N8), and avian Influenza A/Duck/Pennsylvannia/10218/84 (H5N2) in MDCK cells. Thiovir antiviral activity was also evaluated against Influenza B virus. Extracellular virus was monitored by Nucleoprotein A antigen capture ELISA subsequent to infection in the presence or absence of titrated Thiovir. Thiovir shows efficacy against viral strains from avian, human and equine influenza with IC₅₀ concentrations ranging from 29 to 55 μM (Table 1). IC₉₀ concentrations were less than 250 μM for all virus tested, generally about 200 μM. The results suggest that Thiovir possesses broad spectrum anti-influenza activity.

TABLE 1
Inhibition of Influenza: Nucleoprotein Antigen Capture ELISA

| Species | Strain | IC₅₀ (μM) | r² Value |
|---|---|---|---|
| human | A/California/2935/03 (H1N1) | 38 | 0.97 |
| human | A/NorthDakota/2463/03 (H3N2) | 29 | 0.98 |
| equine | A/Equine1/Prague/1/56 (H3N8) | 55 | 0.97 |
| avian | A/Duck/Penn/10218/84 (H5N2) | 32 | 0.99 |
| human | B/1060/03 | 92 | 0.87 |

### Example 2

### Synergistic Inhibition of Influenza A Virus Replication by Thiophosphonoformic Acid in Combination with a Neuraminidase Inhibitor

The following example demonstrates the synergistic inhibition of Influenza A virus replication by TPFA combined with a neuraminidase inhibitor against all strains tested.

Synergistic Activity of TPFA Combined with a Neuraminidase Inhibitor Against Influenza A

Thiovir was combined with neuraminidase inhibitor, oseltamivir phosphate (Tamiflu™) to evaluate potential synergistic anti-influenza activity. MDCK cells were pre-incubated with Thiovir and oseltamivir before addition of influenza virus at MOI 0.01. Extracellular virus was detected by ELISA and data analyzed by median effect principal. Drug interactions were determined using the median effect principle. Combination indices (CI) quantify synergy, summation and antagonism as follows: CI<1 (synergy); CI=1 (summation); CI>1 (antagonism) (Chou, T. C. and Talalay, P. (1984) Adv. Enzyme Regul. 22, 27-55). Since dose-effect relationships of Thiovir or foscarnet with zidovudine are not parallel in the median effect plot, exclusivity of effects cannot be established and data for both mutually exclusive (ME) and mutually nonexclusive (MNE) assumptions are shown. The data indicate that Thiovir combined with oseltamivir has synergistic activity against influenza strains from multiple species with average CI of 0.17 (human) and 0.54 (avian). Combination therapy with two or more drugs that have different modes of action and synergistic activity may have advantages, including increased clinical efficacy, reduced drug dosage and reduction of resistance to a single drug. Our results are consistent with the conclusion that Thiovir and neuraminidase inhibitor combinations offer a promising therapeutic option in the event of an avian influenza pandemic.

TABLE 2
Combination Index Values for Thiovir Combined with Oseltamivir

| | Drug | | Combination Index | | | |
|---|---|---|---|---|---|---|
| | Combination | | Values | | | r2 |
| Virus Strain | (molar ratio) | Model | IC₅₀ | IC₇₅ | IC₉₀ | Value |
| A/Duck/Penn/10218/84 | Thiovir:Oseltamivir | ME | 1.1 | 0.58 | 0.31 | 0.99 |
| (H5N2) | (1:5) | MNE | 1.1 | 0.58 | 0.31 | |
| | Thiovir:Oseltamivir | ME | 0.93 | 0.47 | 0.23 | 0.95 |
| | (1:10) | MNE | 0.93 | 0.47 | 0.23 | |
| A/NorthDakota/2463/03 | Thiovir:Oseltamivir | ME | 0.12 | 0.10 | 0.30 | 0.92 |
| (H3N2) | (1:5) | MNE | 0.12 | 0.10 | 0.30 | |
| | Thiovir:Oseltamivir | ME | 0.32 | 0.25 | 0.47 | 0.96 |
| | (1:10) | MNE | 0.34 | 0.26 | 0.47 | |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of inhibiting influenza viral replication in a subject infected with influenza virus, the method comprising administering to the subject a therapeutically effective amount of a composition comprising thiophosphonoformic acid (TPFA).

2. The method according to claim 1 wherein the influenza virus is Influenza A selected from the group comprising H1N1, H3N2, H3N8, H5N1 and H5N2.

3. The method according to claim 1 wherein the composition comprises the trisodium salt, dihydrate form of TPFA.

4. The method according to claim 1 wherein the composition is administered orally.

5. The method according to claim 1 wherein the composition is administered by parenteral injection.

6. A composition comprising a therapeutically effective amount of thiophosphonoformic acid and a therapeutically effective amount of oseltamivir.

7. The composition of claim 6, wherein the amounts of thiophosphonoformic acid and oseltamivir are sufficient to synergistically lower the viral load of a patient infected with influenza virus.

8. A method of inhibiting influenza viral replication in a subject infected with influenza virus, the method comprising co-administering to the subject a therapeutically effective amount of thiophosphonoformic acid (TPFA) and a therapeutically effective amount of oseltamivir.

9. The method according to claim 8 wherein the influenza virus is Influenza A selected from the group comprising H1N1, H3N2, H3N8, H5N1 and H5N2.

10. The method according to claim 8, wherein the TPFA is the trisodium salt, dihydrate form of TPFA.

11. The method according to claim 8 wherein the TPFA is administered orally.

12. The method according to claim 8 wherein the TPFA is administered by parenteral injection.

13. The method according to claim 8, wherein the TPFA and oseltamivir are administered at the same time.
